(19) United States
(12) Patent Application Publication
(10) Pub. No.: US 2022/0105200 A1
(43) Pub. Date: Apr. 7, 2022

(54) METHODS FOR PREVENTING AND TREATING INFLAMMATION AND INFLAMMATORY DISEASE

(71) Applicants: THE REGENTS OF THE UNIVERSITY OF COLORADO, A BODY CORPORATE, Denver, CO (US); COLORADO SCHOOL OF MINES, Golden, CO (US); UNIVERSITY OF CENTRAL FLORIDA RESEARCH FOUNDATION, INC., Orlando, FL (US)

(72) Inventors: Kenneth Liechty, Denver, CO (US); Carlos Zgheib, Aurora, CO (US); Melissa D. Krebs, Englewood, CO (US); Lindel Dewberry, Aurora, CO (US); Sudipta Seal, Orlando, FL (US)

(21) Appl. No.: 17/428,447

(22) PCT Filed: Feb. 4, 2020
(86) PCT No.: PCT/US2020/016630

§ 371 Date: Aug. 4, 2021

Related U.S. Application Data
(60) Provisional application No. 62/800,988, filed on Feb. 4, 2019.
(60) Provisional application No. 62/905,841, filed on Sep. 25, 2019.

(51) Int. Cl. A61K 47/69; A61K 9/06; A61K 47/36; A61K 47/54; A61K 9/16; A61K 31/728; A61K 38/39; A61K 38/36; A61K 33/244; A61K 47/32

(52) U.S. Cl. CPC A61K 47/6929; A61K 9/06; A61K 47/36; A61K 47/549; A61K 47/32; A61K 31/728; A61K 38/39; A61K 38/363; A61K 33/244; A61K 9/167

(57) ABSTRACT

Methods of treating, reducing the risk of, preventing, or alleviating a symptom of inflammation or inflammatory disease, including wounds, diabetic ulcer, and inflammatory bowel disease, by administration of gel-based delivery particles, such as zwitterionic copolymer cryogels or chitosan microgels, containing cerium oxide nanoparticles.

### TECHNICAL FIELD

This invention relates generally to uses of cerium oxide nanoparticles delivered in gel-based delivery particles for preventing and/or treating inflammation and inflammatory disorders including wounds and gastrointestinal inflammatory disorders.

A sequence listing, titled 20190925_SequenceListing_P282008WO01.txt, created 25 Sep. 2019, and comprising 425 bytes, is submitted as an ASCII text file and hereby incorporated by reference.

### BACKGROUND

Diabetes has reached pandemic proportions worldwide, and complications of diabetes, such as diabetic ulcer and impaired wound healing, represent a significant medical problem, with the annual cost of diabetic lower extremity ulcers alone exceeding 1.5 billion dollars. These chronic wounds result in significant morbidity for individuals including long hospitalizations, prolonged exposure to antibiotics, acute and chronic pain, the need for cumbersome wound care, and restricted mobility. In addition, an ulcer of the lower extremity precedes 84% of all diabetic lower extremity amputations, and is the primary cause for hospitalization among diabetics. Despite the enormous impact of these chronic wounds on both individuals and society, effective therapies are lacking. Thus, the modification, correction, or prevention of diabetes impaired wound healing has far-reaching consequences, both on patient outcomes and on healthcare expenditures.

Normal wound repair and the response to injury follows an orderly and well-defined sequence of events that requires the interaction of many cell types, such as inflammatory cells, fibroblasts, keratinocytes, endothelial cells and progenitor cells, as well as the involvement of many growth factors, extracellular matrix (ECM) proteins, and enzymes. In diabetic wound healing, this complex orchestration of wound healing processes is disrupted and results in impaired healing. Significant improvements in wound healing, including diabetic wound healing, has been seen following therapeutic intervention to alleviate oxidative stress and inflammation.

Inflammatory bowel disease (IBD), specifically ulcerative colitis, is similarly characterized by intestinal inflammation, oxidative stress, and a disrupted mucosal barrier. Because current treatments are largely inefficient to improve symptoms and survival of patients suffering from IBD, such as ulcerative colitis, alternative therapies are urgently needed. The present invention addresses such needs.

### SUMMARY

This disclosure provides methods of treating, reducing the risk of, preventing, or alleviating a symptom of, inflammation or an inflammatory disease or condition in a subject, such as wounds, diabetic ulcers, inflammatory bowel disease, or colitis, by administering to the subject an effective amount of a chitosan microgel or a hydrogel (collectively referred to herein as “gel-based particles” or “gel-based delivery particles”) comprising cerium oxide nanoparticles (also referred to as “CeO₂ nanoparticles,” “nanoceria,” or “CNPs”). The CNP in the gel-based particle formulations of this disclosure may comprise a microRNA (miR or miRNA), which are small, noncoding RNA molecules involved in the posttranscriptional regulation of gene expression. miR regulate the inflammatory response at multiple levels. In particular, miR-146a (SEQ ID NO. 1, having sequence ugagaacugaauuccauggguu) acts as the “molecular brake” on the inflammatory response.

Thus, the CNP in the formulations of this disclosure may comprise miR-146a attached to, or embedded within (i.e., non-covalently associated with) the CNP, such that the miR-146a-conjugated CNPs act as an active agent or therapeutic agent that is incorporated into the gel-based compositions of this disclosure.

This disclosure also relates to the use of a gel-based particle composition comprising the CNP in the manufacture of a medicament for promoting wound repair or treating inflammatory bowel disease in a subject. This disclosure also relates to a pharmaceutical formulation comprising the CNP for promoting wound repair or treating inflammatory bowel disease in a subject.

This disclosure also relates to a pharmaceutical formulation comprising the gel-based CNP compositions for treating, reducing the risk of, preventing, or alleviating a symptom of an inflammatory disease or condition such as wounds, diabetic ulcers, inflammatory bowel disease, or colitis, in a subject.

In accordance with embodiments of the present disclosure, a method for treating inflammation in a subject in need thereof may involve administering to the subject a therapeutically effective amount of a chitosan microgel comprising microRNA-conjugated cerium oxide nanoparticles (CNPs). In some examples, the inflammation is associated with a wound, which may be a diabetic ulcer. In some examples, treating inflammation results in an increased rate of wound closure in the subject compared to the rate of wound closure in an untreated subject.

In some examples, the chitosan microgel is administered topically, intradermally, or intramuscularly to the subject. In some examples, the inflammation is associated with ulcerative colitis or Crohn's disease. In some examples, the chitosan microgel is administered orally or rectally to the subject. In some examples, the chitosan microgel is administered to the subject a plurality of times. In some examples, the chitosan microgel is administered daily to the subject. In some examples, administration of the chitosan microgel treats or prevents oxidative stress in the subject.

In some examples, the microRNA comprises miRNA-146a. In some examples, the surface of the CNPs is coated with one or more biocompatible molecules selected from hyaluronic acid, collagen, and fibrinogen. In some examples, the CNPs have a size range of about 3-5 nm. In some examples, the CNPs are doped with a lanthanide selected from one or more of Europium (Eu), Lanthanum (La), Praseodymium (Pr), Neodymium (Nd), Promethium (Pm), Samarium (Sm), Gadolinium (Gd), Terbium (Tb), Dysprosium (Dy), Homium (Ho), Erbium (Er), Thulium (Tm), Ytterbium (Yb), and Lutetium (Lu).

In accordance with embodiments of the present disclosure, a pharmaceutical composition comprises miRNA146a-conjugated cerium oxide nanoparticles (CNPs) embedded within a chitosan microgel. In some examples, the surface of the CNPs is coated with one or more biocompatible molecules selected from hyaluronic acid, collagen, and fibrinogen. In some examples, the CNPs have a size range of about 3-5 nm. In some examples, the CNPs are doped with a lanthanide selected from one or more of Europium (Eu), Lanthanum (La), Praseodymium (Pr), Neodymium (Nd), Promethium (Pm), Samarium (Sm), Gadolinium (Gd), Terbium (Tb), Dysprosium (Dy), Homium (Ho), Erbium (Er), Thulium (Tm), Ytterbium (Yb), and Lutetium (Lu).

In accordance with embodiments of the present disclosure, a microgel comprises chitosan polymers and a cerium oxide nanoparticle (CNP). In some examples, the CNP comprises a therapeutic agent. In some examples, the therapeutic agent is an anti-inflammatory agent. In some examples, the therapeutic agent is a micro RNA (miRNA). In some examples, the miRNA is miRNA-146a.

In accordance with embodiments of the present disclosure, a method of making an anti-inflammatory chitosan microgel composition comprises forming a composition comprising a plurality of chitosan polymers and a cerium oxide nanoparticle (CNP); and crosslinking the chitosan polymers with genipin to form a chitosan microgel comprising anti-inflammatory CNP.

In some examples, the CNP comprises a therapeutic agent. In some examples, the therapeutic agent is an anti-inflammatory agent. In some examples, the therapeutic agent is a micro RNA (miRNA). In some examples, the miRNA is miRNA-146a.

In accordance with embodiments of the present disclosure, a method for treating inflammation in a subject in need thereof involves administering to the subject a therapeutically effective amount of a zwitterionic gel comprising microRNA-conjugated cerium oxide nanoparticles (CNPs).

In some examples, the inflammation is associated with a wound, which may be a diabetic ulcer. In some examples, treating inflammation in the subject results in an increased rate of wound closure in the subject compared to the rate of wound closure in an untreated subject. In some examples, the zwitterionic gel is administered topically, intradermally, or intramuscularly to the subject. In some examples, the inflammation is associated with ulcerative colitis or Crohn's disease. In some examples, the zwitterionic gel is administered orally or rectally to the subject. In some examples, the zwitterionic gel is administered to the subject a plurality of times. In some examples, administration of the zwitterionic gel treats or prevents oxidative stress in the subject. In some examples, the zwitterionic gel is a zwitterionic cryogel. In some examples, the zwitterionic gel comprises [2-(methacryloyloxy)ethyl]dimethyl-(3-sulfopropyl)ammonium hydroxide (SBMA) and/or 2-hydroxyethyl methacrylate (HEMA) monomers. In some examples, the microRNA comprises miRNA-146a. In some examples, the surface of the CNPs is coated with one or more biocompatible molecules selected from hyaluronic acid, collagen, and fibrinogen. In some examples, the CNPs have a size range of about 3-5 nm. In some examples, the CNPs are doped with a lanthanide selected from one or more of Europium (Eu), Lanthanum (La), Praseodymium (Pr), Neodymium (Nd), Promethium (Pm), Samarium (Sm), Gadolinium (Gd), Terbium (Tb), Dysprosium (Dy), Homium (Ho), Erbium (Er), Thulium (Tm), Ytterbium (Yb), and Lutetium (Lu).

In accordance with embodiments of the present disclosure, a pharmaceutical composition may comprise miRNA146a-conjugated cerium oxide nanoparticles (CNPs) embedded within a zwitterionic hydrogel. In some examples, the surface of the CNPs is coated with one or more biocompatible molecules selected from hyaluronic acid, collagen, and fibrinogen. In some examples, the CNPs have a size range of about 3-5 nm. In some examples, the CNPs are doped with a lanthanide selected from one or more of Europium (Eu), Lanthanum (La), Praseodymium (Pr), Neodymium (Nd), Promethium (Pm), Samarium (Sm), Gadolinium (Gd), Terbium (Tb), Dysprosium (Dy), Homium (Ho), Erbium (Er), Thulium (Tm), Ytterbium (Yb), and Lutetium (Lu).

In accordance with embodiments of the present disclosure, a hydrogel may comprise polymerized zwitterionic monomers selected from the group consisting of sulfobetaine methacrylate (SBMA), carboxybetaine methacrylate (CBMA), and combinations thereof. The hydrogel may further comprise polymerized hydroxyethyl methacrylate (HEMA) monomers and a cerium oxide nanoparticle (CNP).

In some examples, the CNP comprises a therapeutic agent. In some examples, the therapeutic agent is an anti-inflammatory agent. In some examples, the therapeutic agent is a micro RNA (miRNA). In some examples, the miRNA is miRNA-146a.

In accordance with embodiments of the present disclosure, a method of making an anti-inflammatory hydrogel composition may involve forming a composition comprising at least one zwitterionic monomer selected from the group consisting of sulfobetaine methacrylate (SBMA) and carboxybetaine methacrylate (CBMA); hydroxyethyl methacrylate (HEMA) monomers; and a cerium oxide nanoparticle (CNP). The method may further comprise initiating the polymerization of the monomers in the composition by the addition of a chemical polymerizing agent to the composition, and polymerizing the monomers in the composition at a temperature below 0° C. to form a hydrogel comprising anti-inflammatory CNP.

In some examples, the CNP comprises a therapeutic agent. In some examples, the therapeutic agent is an anti-inflammatory agent. In some examples, the therapeutic agent is a micro RNA (miRNA). In some examples, the miRNA is miRNA-146a. In some examples, the polymerizing agent comprises ammonium persulfate (APS) and N,N,N′,N′-Tetramethylethylenediamine (TEMED). In some examples, the step of polymerizing the monomers in the composition is conducted at a temperature of about −20° C.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Detailed Description and no limitation as to the scope of the present disclosure is intended by either the inclusion or non-inclusion of elements, components, etc. in this Summary. Other features and advantages of the invention will be apparent from the following detailed description and claims.

### BRIEF DESCRIPTION OF FIGURES

FIG. 1 shows FTIR spectra of the dried zwitterionic copolymer cryogels (a) SBMA, and (b) CBMA.

FIG. 2 shows rheology results of the zwitterionic cryogels prepared using 1:1 mole ratio of zwitterionic (CBMA or SBMA) and non-zwitterionic monomer (HEMA).

FIG. 3 shows rheology results of the cryogels prepared using different mole ratios of monomers.

FIG. 4 shows rheology results for the zwitterionic gels polymerized at cryoconditions or room temperature.

FIG. 5 shows GPC results for a hydrogel of this disclosure created by polymerization at either room temperature (RTgel) or cryogelation at −20C (Cryogel).

FIG. 6 shows rheology of the CH1c samples after additional freeze thaw cycles.

FIG. 7 shows the swelling (gain in mass over time) for two cryogels of this disclosure.

FIG. 8 shows the degradation (loss in mass over time) for two cryogels of this disclosure.

FIG. 9 shows the effects of topical application of cryogels of this disclosure (with or without CNP-miR146a) on time to complete wound healing in diabetic mice with skin wounds.

FIG. 10 shows the results of stress testing of mouse skin treated with cryogels of this disclosure (with or without CNP-miR146a).

FIG. 11 shows the gene expression of miR146a using various CNP-miR146a delivery vehicles.

FIG. 12 shows the Disease Activity Index (DAI) over time between DSS mice treated with CNP-miR146a gel, control gel, and no treatment.

FIG. 13 shows the weight loss over time between DSS mice treated with CNP-miR146a chitosan microgel, chitosan microgel with no CNP-miR146a, control gel, and no treatment.

FIG. 14 shows the DAI over time between DSS mice treated with CNP-miR146a chitosan microgel, chitosan microgel with no CNP-miR146a, control gel, and no treatment.

FIG. 15 shows the colon length of DSS mice treated with CNP-miR146a chitosan microgel, chitosan microgel with no CNP-miR146a, control gel, and no treatment.

FIG. 16 shows the gene expression of IL-1b in DSS mice after 2 days of treatment with CNP-miR146a chitosan microgel, chitosan microgel with no CNP-miR146a, control gel, and no treatment.

FIG. 17 shows the gene expression of TNFα in DSS mice after treatment with CNP-miR146a chitosan microgel and chitosan microgel with no CNP-miR146a.

### DETAILED DESCRIPTION

This disclosure relates to a method of treating, reducing the risk of, preventing, or alleviating symptoms of wounds, including diabetic wounds, and inflammatory bowel disease (IBD), by administering gel-based particle formulations loaded with cerium oxide nanoparticles to a subject in need of such treatment. Specific examples include gel-based particles comprising zwitterionic cyrogels or chitosan microgels.

Unless otherwise defined, all technical and scientific terms used herein have the same meaning as commonly understood by one of ordinary skill in the art to which this invention belongs. In the case of conflict, the present specification, including definitions, will control. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference. The references cited herein are not admitted to be prior art to the claimed invention. In addition, the materials, methods, and examples are illustrative only and are not intended to be limiting.

Therapeutic Compositions

The therapeutic compositions of this disclosure, which are particularly useful in the therapeutic methods of this disclosure, include gel-based delivery particles comprising anti-inflammatory therapeutic agents. The anti-inflammatory therapeutic agents may be covalently linked to the components of the gel-based particles or non-covalently entrapped or embedded in the gel-based particles. Preferably, the anti-inflammatory therapeutic agents are held non-covalently within the gel-based particles and released to the site of administration to a subject treated with the gel-based particle.

The gel-based particles can comprise chitosan microgels or hydrogels. The chitosan microgels include chitosan molecules, which are linear polysaccharide polymers comprised of linked glucosamine and N-acetyl-glucosamine monomers. The chitosan microgels are three-dimensional networks of chitosan molecules that may be crosslinked, bonded or otherwise coupled by various techniques. Crosslinked chitosan microgels can be crosslinked, for example with genipin, by various physical and/or chemical methods. The chitosan microgels can be loaded with one or more anti-inflammatory therapeutic agents before, during or after microgel formation. Characteristics (e.g., loading, elasticity, porosity, biodegradation rate, viscosity, antifouling properties, etc.) of these microgels may be modified by varying the concentration of chitosan polymer subunits.

The chitosan microgels can form a solid or semi-solid scaffold, gel, film, or coating comprising the anti-inflammatory therapeutic agent(s) disclosed herein. The chitosan microgels may retain cargo, e.g., CNPs, for greater lengths of time, for example relative to the hydrogel formulations described herein. Example microgel formulations may also be configured to contain greater concentrations of cargo, e.g., CNPs, relative to the disclosed hydrogels and/or other delivery particles. The chitosan microgels may also exhibit a higher melting temperature and/or enhanced cohesion, adhesion and/or general “stickiness” relative to the hydrogels disclosed herein. Due at least in part to the aforementioned improvements, use of the chitosan microgels may also result in a more consistent reduction in the expression of inflammation-implicated gene markers. The chitosan microgels may be absorbent and may possess excellent antifouling properties and biocompatibility. They may also demonstrate self-healing properties. Additionally, embodiments of the microgels comprising relatively lower amounts of the chitosan polymers may be softer and therefore may be suitable for administration by injection.

Methods of forming the microgels may vary. For example, in some embodiments, the microgels can be formed by crosslinking chitosan polymers with a crosslinking agent, such as genipin. Specific examples of such embodiments may involve first dissolving purified chitosan in acetic acid, for e.g., about 2.4 grams of chitosan in about 40 mL of 6% acetic acid. The mixture can then be stirred, for e.g., at about 200 rpm, in a temperature-controlled water bath, for e.g., at 40° C., for about 3 hours, or until all the chitosan is completely dissolved. While stirring, a crosslinking agent, e.g., genipin, can be added. In some examples, about 2 mL of 100 mM genipin in absolute ethanol can be used for this step. The chitosan-genipin solution can then be mixed with a stir bar, uncovered, for at least about 10 minutes, at which point PARAFILM can be applied to prevent evaporation. The covered solution can be mixed for an extended period, for e.g., overnight, at the same or similar temperature, for e.g., about 40° C. After this mixing period, the newly-formed, crosslinked gel can be transferred to a sieve, for e.g., a 106 μm sieve. The gel can be manually pressed through the sieve and the separated microgel particles collected. Periodic addition of water may facilitate microgel particle separation during this step. The collected microgels can then be passed through the sieve one or more additional times to ensure formation of uniformly sized particles. Particularly large particles, for e.g., >250 μm, can be separated from the collection of microgel particles by shaking the collection through a 250 μm sieve while rinsing with water. Vacuum filtration can then be used to remove any excess water. The microgel particles can then be resuspended in an appropriate media for application. Swelling and de-swelling can be allowed to occur until the particles reach a state of equilibrium, after which the final suspension can be centrifuged one or more times, for e.g., 3 times at about 2000×g for about 5 minutes in 50 mL falcon tubes. The supernatant is decanted after each centrifugation and the collection of microgel particles stored. When ready to use, the microgels can be centrifuged again and then resuspended in a suspension medium to produce a 1:1 dilution.

In additional embodiments, an emulsion-based method can be used to form the chitosan microgels. Such methods may involve crosslinking a mixture of chitosan and genipin as a stable emulsion, removing the oil phase therefrom, and adjusting the pH, for example as described by Michael S. Riederer et al. in “Injectable and microporous scaffold of densely-packed, growth factor-encapsulating chitosan microgels,” Carbohydrate Polymers, 152: 792-801 (2016), which is incorporated by reference in its entirety herein.

The hydrogel formulations of this disclosure are three-dimensional networks of monomers that in some examples may be crosslinked by physical and/or chemical methods. The monomers may have two or more charged groups over a given pH range. In embodiments, the polymers comprise zwitterionic monomers. As used herein, a zwitterionic monomer is any compound that is able to be polymerized and simultaneously includes both a positively and negatively charged group under physiological conditions. Characteristics (e.g., loading, elasticity, porosity, biodegradation rate, viscosity, antifouling properties, etc.) of these hydrogels may be modified by varying the concentration of monomer subunits.

Example hydrogels can be formed as copolymers comprising one or both of the zwitterionic monomers sulfobetaine methacrylate (SBMA), and/or carboxybetaine methacrylate (CBMA), and the non-zwitterionic monomer hydroxyethyl methacrylate (HEMA). One or more anti-inflammatory therapeutic agents can be added to the composition of monomers prior to polymerization in some examples. In embodiments, the monomers can be polymerized in the absence of any chemical crosslinker, for example as described by Gulsu Sener et al. in “Injectable, self-healable zwitterionic cryogels with sustained microRNA—cerium oxide nanoparticle release promote accelerated wound healing,” Acta Biomaterialia, 101: 262-272 (2020), which is incorporated by reference in its entirety herein. The polymerization of the monomers can be initiated with the addition of ammonium persulfate (APS) and N,N,N′,N′-Tetramethylethylenediamine (TEMED).

Hydrogel polymerization can be performed in a temperature range between about room temperature and about −20° C. When the polymerization is performed at a temperature below the freezing point of the aqueous phase of the gel solution, the resulting hydrogel may be referred to as a “cryogel” or “cryotopic gel.” Thus, these cryogels may be synthesized in semi-frozen liquid media in which ice crystals forming in the media act as porogen (pore generator) to create interconnected macro-pores after thawing. The shape and size of the ice crystals may help to modify the morphology and the porosity of the resulting cryogel. The polymerization temperature may be any temperature below the freezing point of the aqueous phase of the gel solution. Factors such as ratio of monomer subunits, polymerization temperature, rate of freezing, and solvent composition may be used to modify the characteristics of the cryogel. For example, the internal pore size and density of the cryogel may be varied by modifying the ratio of monomer subunits and the polymerization temperature. In embodiments, the average pore size of the disclosed cryogels may be between about 50 μm and about 100 μm.

Hydrogel polymerization can be conducted at about −20° C. (thereby preparing cryogels). Polymerization may proceed for a time period between about 1 hour and about 24 hours. In specific embodiments, polymerization can be conducted at a temperature of about −20° C. for a time period of about 24 hours. After polymerization, the cryogels may be thawed at room temperature. The mole ratio of the zwitterionic monomer (SBMA and/or CBMA) to the non-zwitterionic monomer (HEMA) may range from about 50:1 to about 1:50. For example, the mole ratio of zwitterionic to non-zwitterionic monomer may be about 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50 or between any two listed values (i.e. between about 1:3 and 1:4, or between about 2:1, and 1:2). The mole ratio of the zwitterionic monomer (SBMA and/or CBMA) to the non-zwitterionic monomer (HEMA) can be about 1:1. In some embodiments, the total concentration of monomers in a gel may vary from about 30 mg/ml or less to about 230 mg/ml or more, for example more than about 25 mg/ml, 30 mg/ml, 40 mg/ml, 45 mg/ml, 50 mg/ml, 55 mg/ml, 60 mg/ml, 65 mg/ml, 70 mg/ml, 75 mg/ml, 80 mg/ml, 85 mg/ml, 90 mg/ml, 95 mg/ml, 100 mg/ml, 110 mg/ml, 120 mg/ml, 130 mg/m1,.140 mg/ml, 150 mg/ml, 160 mg/ml, 170 mg/ml, 180 mg/ml, 190 mg/ml, 200 mg/ml, 210 mg/ml, or 220 mg/ml, and less than about 250 mg/ml, 240 mg/ml, 230 mg/ml, 220 mg/ml, 210 mg/ml, 200 mg/ml, 190 mg/ml, 180 mg/ml, 170 mg/ml, 160 mg/ml, 150 mg/ml, 140 mg/ml, 130 mg/ml, 120 mg/ml, 110 mg/ml, 100 mg/ml, 90 mg/ml, 80 mg/ml, 70 mg/ml, 60 mg/ml, 50 mg/ml, 40 mg/ml, or 30 mg/ml. The polymerized hydrogels possess a macroporous structure with interconnected pores. In some examples, the cryogels may be formed in the absence of any chemical crosslinker, which may result in cryogels that are less brittle than cryogels formed with a cross-linking agent.

Following polymerization, these hydrogels may form a solid or semi-solid scaffold, gel, film, or coating comprising the anti-inflammatory therapeutic agent(s). These hydrogels may be absorbent and may possess excellent antifouling properties and biocompatibility. The biocompatibility may be due to the high-water content and physiochemical similarity of the hydrogels to native extracellular networks. These cryogels formed from zwitterionic monomer (SBMA and/or CBMA) and a non-zwitterionic monomer (HEMA) are found to be mechanically stable after stretching or compression. They also demonstrate self-healing properties, rendering them resilient to physical stresses and/or damage by enabling them to repair themselves, partially or completely. Additionally, hydrogels comprising lower amounts of the zwitterionic monomers are softer and therefore may be suitable for administration by injection.

An exemplary anti-inflammatory therapeutic agent for incorporation into the gel-based particles described above is a cerium oxide nanoparticle (also referred to as “CeO₂ nanoparticles,” “nanoceria,” or “CNP”). These CNPs are especially useful active agents in the pharmaceutical formulations of this disclosure. The production of such cerium oxide nanoparticles has been described in, for example, Chigurupati, et al., Biomaterials 34(9):2194-2201 (2013); and U.S. Pat. No. 7,534,453, which is incorporated herein by reference in its entirety. The CNPs in these pharmaceutical formulations may have a size range of about 2-10nm, and in particular about 3-5 nm. These CNPs may be covalently conjugated to, or otherwise incorporated (i.e., non-covalently embedded in or associated with), additional therapeutic agents (for example, micro RNA molecules, as described below).

Another useful active agent in these gel-based particle formulations is microRNA (miR or miRNA), which are small noncoding RNA molecules involved in the post transcriptional regulation of gene expression. miR regulate the inflammatory response at multiple levels. In particular, miR-146a acts as the “molecular brake” on the inflammatory response, by targeting and repressing the activation of the NFκB inflammatory pathway. Thus, the gel-based formulations of this disclosure may comprise miR-146a (SEQ ID NO. 1). These miR-146a active agents may be further conjugated to the CNPs described above, such that the miR-146a-conjugated CNPs (“CNP-146a”) act as an active agent or therapeutic agent in the gel-based formulations of this disclosure. The detailed synthesis and characterization of CNP conjugated to miRNA-146a has been described in PCT Publication No. WO 2017/091700 with international filing date of 23 Nov. 2016, which is incorporated herein by reference. Briefly, oligonucleotides (i.e. miRNA-146a) contain phosphate groups carrying a negative charge along the chain that can interact electrostatically with the positively-charged surface of the CNPs. In addition, oligonucleotides have hydroxyl groups of ribose and amino groups available for conjugation with the CNPs. The terminal functional group (amino, thiol, azide) for conjugation is also an option. Providing an appropriate excess of oligonucleotide in reaction medium (basically 10 -15 molecules per nanoparticle), conjugation can be accomplished via different reactions. For example, amino groups of an oligonucleotide can be coupled with CNP hydroxyl groups or functional groups of CNP coating after their activation with carbodiimide (CDI), or other bifunctional activating agents. Unbound compounds, as well as by-products, can be removed by centrifugation at 8000 g for 10 min and by dialysis against water or PBS using mini dialysis columns with at least 20kDa cut off.

CNP-146a may be incorporated into the gel-based particles by addition of the CNP-146a to a composition of chitosan polymers or, in embodiments comprising hydrogel formulations, to the composition of monomers (zwitterionic monomer (SBMA and/or CBMA) and a non-zwitterionic monomer (HEMA)) prior to polymerization (which is preferably cyrogelation, as described above) to form a gel-based particle loaded with an effective amount of CNP-146a that is released from the gel-based particle at the site of administration of the gel-based particle on the subject.

An “effective amount” of a CNP-146a composition of this disclosure, is an amount sufficient to carry out a specifically stated purpose. An “effective amount” may be determined empirically and in a routine manner, in relation to the stated purpose. The term “therapeutically effective amount” refers to an amount of a CNP composition, to “treat” a disease or disorder in a subject.

As used herein, “promoting” or “promote” means reducing the time for the skin or intestinal mucosa to repair or recover from injuries or inflammation to the skin or mucosa of the colon increasing the extent of tissue repair or recovery. These formulations may promote repair or recovery by reducing or suppressing inflammation in the epithelial or mucosal tissues.

As used herein, “suppressing”, “suppress”, or “suppression” means stopping the inflammation from occurring, worsening, persisting, lasting, or recurring.

“Reducing”, “reduce”, or “reduction” means decreasing the severity, frequency, or length of the inflammation.

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for an inflammatory disease or disorder if, after receiving a therapeutic amount of a CNP composition, according to the methods of this disclosure, the subject shows observable and/or measurable reduction in, or absence of, one or more symptoms of the inflammatory disease or disorder or increased rates of healing or repair. Reduction of these signs or symptoms may also be felt by the patient.

Therapeutic Formulations

Gel-based CNP-146a compositions of this disclosure may be administered as a pharmaceutical formulation. The CNP-146a gel-based compositions of this disclosure may comprise one or more pharmaceutically-acceptable excipients and are typically formulated into a dosage form adapted for topical, rectal, or oral administration to a subject.

A “subject” herein is typically a human. In certain embodiments, a subject is a non-human mammal. Exemplary non-human mammals include laboratory, domestic, pet, sport, and stock animals, e.g., mice, cats, dogs, horses, and cows. Typically, the subject is eligible for treatment, e.g., treatment of a gastrointestinal inflammatory disorder. As used herein, the term “patient” refers to any single subject for which treatment is desired. In certain embodiments, the patient herein is a human. A subject can be considered to be in need of treatment.

As used herein, a “pharmaceutically-acceptable excipient” or a “pharmaceutically-acceptable carrier” means a pharmaceutically acceptable material, composition, or vehicle involved in giving form or consistency to the pharmaceutical composition. Each excipient or carrier must be compatible with the other ingredients of the pharmaceutical composition when comingled such that interactions which would substantially reduce the efficacy of the active CNP compositions of this disclosure when administered to a subject and interactions which would result in pharmaceutical compositions that are not pharmaceutically acceptable are avoided. In addition, each excipient or carrier must of course be of sufficiently high purity to render it pharmaceutically-acceptable. Suitable pharmaceutically acceptable excipients will vary depending upon the particular dosage form chosen, the particular function that they serve in the compositions, their ability to facilitate the production of stable dosage forms, and/or to enhance patient compliance. Suitable pharmaceutically acceptable excipients may include diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the gel-based formulations of this disclosure. In addition, there are resources available to the skilled artisan which describe pharmaceutically-acceptable excipients and may be useful in selecting suitable excipients for use in the gel-based compositions of this disclosure. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

Therapeutic formulations comprising the gel-based CNP-146a compositions of this disclosure may be prepared for storage by mixing the gel-based CNP-146a composition having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations.

Sustained-release preparations may be prepared. Suitable examples of sustained-release preparations may include semipermeable matrices of solid hydrophobic polymers containing the gel-based CNP-146a compositions of this disclosure in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days.

Thus, one aspect of this disclosure is a therapeutic formulation comprising the gel-based CNP-146a composition of this disclosure adapted for topical administration to a subject. Such topical formulations are particularly useful in the methods of treating, preventing, or alleviating wounds, including diabetic wounds, of this disclosure. The therapeutic formulation may comprise an appropriate dosage form for topical administration, such as a gel, cream, ointment, salve, or medicated bandage comprising the gel-based CNP-146a compositions of this disclosure.

Another aspect of this disclosure is a therapeutic formulation comprising the gel-based CNP-146a composition of this disclosure adapted for rectal administration to a subject. Such rectal formulations are particularly useful in the methods of treating, preventing, or alleviating inflammatory bowel diseases, e.g., colitis, of this disclosure. The therapeutic formulation may comprise an appropriate dosage forms for rectal administration, such as a gel, suspension, or solution comprising the gel-based CNP-146a compositions of this disclosure.

Another aspect of this disclosure is a therapeutic formulation comprising the gel-based CNP-146a composition of this disclosure adapted for oral administration to a subject. Such oral formulations are particularly useful in the methods of treating, preventing, or alleviating inflammatory bowel diseases, of this disclosure. Appropriate dosage forms for oral administration, such as a tablet, capsule, solution, or suspension comprising the gel-based CNP-146a compositions may be prepared by conventional techniques.

Therapeutic Methods

The gel-based CNP-146a compositions of this disclosure are suitable for the treatment of, reducing the risk of, prevention of, or alleviation of a symptom of a variety of inflammatory diseases or conditions, such as colitis. Without intending to be bound by theory, CNP compositions of this disclosure are reactive oxygen species scavengers and are rapidly taken up by epithelial cells, decreasing inflammation and/or suppressing the movement of leukocytes or fibrocytes from circulation to inflamed tissues. These CNP compositions may also improve cell viability and cell regeneration.

The pharmaceutical formulations of this disclosure are suitable for treating, reducing the risk of, preventing, or alleviating a symptom of inflammatory skin diseases or conditions caused by or associated with inflammation, such as inflammatory bowel disease.

A. Wound Healing

Impaired wound healing following injury in diabetic subjects represents a major clinical problem, resulting in prolonged hospitalizations and significant healthcare expenditures. Two-thirds of all non-traumatic amputations are preceded by a diabetic wound. The impaired healing of diabetic wounds is multifactorial and has been characterized by decreased production of chemokines, decreased angiogenesis, and an abnormal inflammatory response. Increasing evidence suggests that the persistent up-regulation of inflammatory gene expression may contribute to the pathogenesis of the chronic diabetic wounds through activation of inflammatory pathways. One of the mechanisms of mesenchymal stem cell (MSC) correction of the diabetic wound healing impairment is through decreasing the inflammatory response.

Inflammation is an important component of normal wound healing. However, increased or persistent inflammation results in the accumulation of reactive oxygen species (ROS) and increased oxidative stress. The CNP present in the gel-based CNP-146a compositions of this disclosure may scavenge excess ROS, similar to the catalytic activity of superoxide dismutase (SOD) and catalase. The CNPs are therefore useful in methods to accelerate the healing of wounds in a subject in need thereof compared to the rate of wound healing in an untreated subject. The CNPs are also useful in methods to accelerate healing of excisional wounds in a subject in need thereof compared to the rate of wound healing in an untreated subject.

The regulation of the inflammatory response occurs at multiple levels, and, as described above, miR-146a decreases or prevents the inflammatory response by targeting and repressing the activation of the NFκB inflammatory pathway. Chronic inflammation has been implicated as a major component in the pathogenesis of the diabetic wound healing impairment by increasing oxidative stress in the wound. It was observed that microRNA-146a and its targeted pro-inflammatory signaling pathways are dysregulated in diabetic subjects, resulting in increased and persistent inflammation. Expression of miR-146a is significantly down-regulated in diabetic wounds and MSC correction of the wound healing impairment is associated with increased miR-146a expression and down-regulation of inflammatory cytokine production. Thus, the gel-based CNP-146a compositions of this disclosure are useful in methods for the treatment of a wound in a subject in need of such treatment. In these methods, the wound may be a diabetic wound. In these methods, the administration of gel-based CNP-146a compositions of this disclosure decrease the area of the diabetic wound, similar to the size of a non-diabetic wound at 7 and 10 days. In these methods, administration of the gel-based CNP-146a compositions of this disclosure may decrease the inflammatory response, resulting in decreased ROS and oxidative stress and lead to improved diabetic wound healing.

In addition to impaired wound healing following injury, diabetics are predisposed to injury and the development of a chronic non-healing wound. Two-thirds of all non-traumatic amputations in the US are preceded by a diabetic foot wound. A significant factor that predisposes the diabetic to injury is the development of peripheral neuropathy, which affects up to 50% of patients with diabetes, resulting in altered perception of thermal, tactile, and vibrational stimuli. This has led to a focus on preventative measures to minimize foot damage in diabetic patients and decrease the incidence of non-healing diabetic wounds. However, many studies have demonstrated that physicians and patients are poorly compliant with simple foot care assessment programs. Previous research has focused on potential impairments in the diabetic skin at baseline that may predispose the diabetic skin to injury. In studies in which the biomechanical properties of diabetic skin in mice and humans at baseline were examined, both murine and human diabetic skin demonstrated impaired skin integrity, with significantly inferior biomechanical properties at baseline. Both Max Stress and modulus were significantly decreased compared to non-diabetic skin, which may predispose the diabetic skin to injury. Correction of these biomechanical properties of diabetic skin at baseline may decrease the susceptibility of diabetic skin to injury and aid in the prevention of the development of a chronic wound. Thus, the gel-based CNP-146a compositions of this disclosure are also useful in methods for decreasing the susceptibility of diabetic skin to injury and/or aiding in the prevention of the development of a chronic wound by topical administration to a subject in need of such treatment.

B. Inflammatory Gastrointestinal Disorders

“Gastrointestinal inflammatory disorders” are a group of chronic disorders that cause inflammation and/or ulceration in the mucous membrane. These disorders include, for example, inflammatory bowel disease (e.g., Crohn's disease, ulcerative colitis, indeterminate colitis and infectious colitis), mucositis (e.g., oral mucositis, gastrointestinal mucositis, nasal mucositis and proctitis), necrotizing enterocolitis and esophagitis. Inflammatory Bowel Disease (IBD) is used interchangeably herein to refer to diseases of the bowel that cause inflammation and/or ulceration and includes without limitation Crohn's disease and ulcerative colitis. Crohn's disease (CD) and ulcerative colitis (UC) are chronic inflammatory bowel diseases of unknown etiology. Crohn's disease, unlike ulcerative colitis, can affect any part of the bowel. The most prominent feature of Crohn's disease is the granular, reddish-purple edematous thickening of the bowel wall. With the development of inflammation, these is granulomas often lose their circumscribed borders and integrate with the surrounding tissue. Diarrhea and obstruction of the bowel are the predominant clinical features. As with ulcerative colitis, the course of Crohn's disease may be continuous or relapsing, mild or severe, but unlike ulcerative colitis, Crohn's disease is not curable by resection of the involved segment of bowel. Most patients with Crohn's disease require surgery at some point, but subsequent relapse is common and continuous medical treatment is usual.

Crohn's disease may involve any part of the alimentary tract from the mouth to the anus, although typically it appears in the ileocolic, small-intestinal or colonic-anorectal regions. Histopathologically, the disease manifests by discontinuous granulomatomas, crypt abscesses, fissures and aphthous ulcers. The inflammatory infiltrate is mixed, consisting of lymphocytes (both T and B cells), plasma cells, macrophages, and neutrophils. There is a disproportionate increase in IgM- and IgG-secreting plasma cells, macrophages and neutrophils.

Anti-inflammatory drugs sulfasalazine and 5-aminosalisylic acid (5-ASA) are used for treating mildly active colonic Crohn's disease and are commonly prescribed in an attempt to maintain remission of the disease. Metroidazole and ciprofloxacin are similar in efficacy to sulfasalazine and are particularly prescribed for treating perianal disease. In more severe cases, corticosteroids are prescribed to treat active exacerbations and can sometimes maintain remission. Azathioprine and 6-mercaptopurine have also been used in patients who require chronic administration of corticosteroids. It has been suggested that these drugs may play a role in the long-term prophylaxis. Unfortunately, there can be a very long delay (up to six months) before onset of action in some patients. Antidiarrheal drugs can also provide symptomatic relief in some patients. Nutritional therapy or elemental diet can improve the nutritional status of patients and induce symptomatic improvement of acute disease, but it does not induce sustained clinical remissions. Antibiotics are used in treating secondary small bowel bacterial overgrowth and in treatment of pyogenic complications.

Ulcerative colitis (UC) afflicts the large intestine. The course of the disease may be continuous or relapsing, mild or severe. The earliest lesion is an inflammatory infiltration with abscess formation at the base of the crypts of Lieberkuhn. Coalescence of these distended and ruptured crypts tends to separate the overlying mucosa from its blood supply, leading to ulceration. Symptoms of the disease include cramping, lower abdominal pain, rectal bleeding, and frequent, loose discharges consisting mainly of blood, pus, and mucus with scanty fecal particles. A total colectomy may be required for acute, severe or chronic, unremitting ulcerative colitis. The clinical features of UC are highly variable, and the onset may be insidious or abrupt, and may include diarrhea, tenesmus and relapsing rectal bleeding. With fulminant involvement of the entire colon, toxic megacolon, a life-threatening emergency, may occur. Extraintestinal manifestations include arthritis, pyoderma gangrenoum, uveitis, and erythema nodosum.

Treatment for UC includes sulfasalazine and related salicylate-containing drugs for mild cases and corticosteroid drugs in severe cases. Topical administration of either salicylates or corticosteroids is sometimes effective, particularly when the disease is limited to the distal bowel and is associated with decreased side effects compared with systemic use. Supportive measures such as administration of iron and antidiarrheal agents are sometimes indicated. Azathioprine, 6-mercaptopurine and methotrexate are sometimes also prescribed for use in refractory corticosteroid-dependent cases.

Monoclonal antibodies targeting tumor necrosis factor alpha (TNF-α), such as infliximab (a chimeric antibody) and adalimumab (a fully human antibody), are also used in the management of CD. Infliximab has also shown efficacy and has been approved for use in UC. However, approximately 10%-20% of patients with CD are primary non-responders to anti TNF therapy, and about 20%-30% of CD patients lose response over time. Other adverse events associated with anti-TNF therapies include elevated rates of bacterial infection, including tuberculosis, and, more rarely, lymphoma and demyelination. In addition, most patients do not achieve sustained steroid-free remission and mucosal healing, clinical outcomes that correlate with true disease modification. Therefore, there is a need for therapy in IBD that is optimized for chronic use, including improved safety profiles with sustained remission, particularly steroid-free remission and prevention of long-term complications in a greater proportion of patients, including those patients who either never respond to an anti-TNF therapeutic agent or lose response over time.

Similar to the role of inflammation in slowing or preventing the healing of skin wounds, inflammatory bowel disease (IBD), specifically ulcerative colitis, is characterized by intestinal inflammation, and oxidative stress. Therefore, administration of the gel-based CNP-146a compositions of this disclosure to target oxidative stress can be used to treat inflammatory bowel diseases by reducing inflammation and oxidative stress in the intestinal mucosa.

The gel-based CNP-146a compositions of this disclosure may be administered orally or rectally on a chronic or intermittent basis and are suitable for treating, reducing the risk of, preventing, or alleviating a symptom of inflammatory bowel diseases, including ulcerative colitis, indeterminate colitis, and Crohn's disease. In these methods, the response to administration of the gel-based CNP-146a compositions of this disclosure may include one or more of clinical response, mucosal healing, and remission.

To bring the gel-based CNP-146a compositions into contact with the inflamed intestinal mucosa, these gel-based CNP-146a compositions may be formulated for oral or rectal administration, as described above. In these methods, oral and/or rectal administration of a therapeutic formulation comprising the gel-based CNP-146a composition of this disclosure may be useful in treating, preventing, or alleviating inflammatory bowel diseases.

C. Administration

In these therapeutic methods of this disclosure, the clinician administering treatment will be able to determine the appropriate dose for the individual subject for weight-based or flat dosing (i.e., a particular amount of the gel-based CNP-146a composition that is administered to every patient regardless of weight). For the prevention or treatment of disease, the appropriate dosage of the gel-based CNP-146a compositions and any second therapeutic or other compound administered in combination with the gel-based CNP-146a compositions may depend on the disease state being treated, e.g., the type of wound to be treated or the gastrointestinal inflammatory disorder to be treated (IBD, UC, CD) the severity and course of the disease, whether the gel-based CNP-146a composition or combination is administered for preventive or therapeutic purposes, previous therapy, the patient's clinical history and response to the CNP-146a hydrogen or chitosan microgel, and the discretion of the clinician. In these methods, the gel-based CNP-146a compositions can be suitably administered to the patient at one time or more typically over a series of treatments. For example, the gel-based CNP-146a compositions may be administered once every week, or once every two weeks, or once every four weeks, or once every six weeks, or once every eight weeks for a period of one month (4 weeks), or two months, three months, or six months, or 12 months, or 18 months, or 24 months, or chronically for the lifetime of the patient. Alternatively or additionally, the gel-based CNP-146a composition treatments may be self-administered by the patient. For repeated administrations over several days or longer, depending on the condition, the treatment can be sustained until a desired suppression of disease symptoms occurs. However, other dosage regimens may be useful. Typically, the clinician will administer a gel-based CNP-146a composition of this disclosure (alone or in combination with a second compound) of the invention until a dosage(s) is reached that provides the required biological effect. The progress of the therapy of the invention is easily monitored by conventional techniques and assays.

As described above, the gel-based CNP-146a composition can be administered by any suitable means, including topical, intralesional, oral, and/or rectal administration.

The foregoing written specification and following examples are considered to be sufficient to enable one skilled in the art to practice the invention. Various modifications of the invention in addition to those shown and described herein will become apparent to those skilled in the art from the foregoing description and following examples and fall within the scope of the appended claims. The disclosures of all citations in the specification are expressly incorporated herein by reference.

### EXAMPLES

### Example 1

Preparation of CNP-146a Hydrogel Compositions from Zwitterionic Monomers

Copolymer hydrogels were prepared from different combinations of two different zwitterionic monomers. The following materials and methods were used to make and test the hydrogel compositions:

Materials: [2-(methacryloloxy)ethyl]dimethyl-(3-sulfopropyl) amomonium hydroxide (SBMA) was purchased from AFG Bioscience, 3-[[2-(Methacryloyloxy)ethyl]dimethylammonio]propionate (CBMA) was purchased from TCI America, 2-Hydroxyethyl methacrylate (HEMA), ammonium persulfate (APS), and N,N,N,N-tetramethylethylenediamine (TEMED) were purchased from Acros Organics). Phosphate buffered saline (PBS) with calcium and magnesium was purchased from HyClone. All chemicals were used as received.

Gel preparation: Hydrogels were prepared by dissolving appropriate amounts of SBMA or CBMA and 2-hydroxyethyl methacrylate (HEMA) in 0.45 mL water. The monomer concentrations used in this study are given in Tables 1 and 2. Polymerization was initiated using 50 μL of 13.6 mg/mL APS solution and 0.85 μL TEMED, and the reaction mixtures were poured into plastic molds (3 mL syringe with inner diameter 0.5 cm or 12 well tissue culture plate) and polymerized at room temperature or −20° C. for 24 hr. Cryogels were thawed at room temperature after polymerization was complete.

Characterization: For rheology measurements, gel samples of about 22 mm diameters and about 4 mm in height were prepared in 12 well plates. To determine the rheological properties of the gels, frequency sweep and strain sweeps test were performed using an AR-G2 rheometer (TA Instruments) equipped with a 20mm diameter crosshatched parallel plate at 37° C. Frequency sweep measurements were performed at 1% strain. Strain sweep measurements were performed at 10 rad/s frequency. For FTIR measurements gel samples were lyophilized and dried powders were analyzed using a Nexus 470 ESP FT-IR Spectrometer equipped with an ATR accessory (Specac, Golden Gate).

Swelling tests: To determine the swelling behavior of the cryogels, as-prepared gels were weighed to determine the mass of initial samples (mᵢₙᵢₜᵢₐₗ) soaked in PBS (pH:7.4), and allowed to swell in an incubator at 37° C. The hydrogels were taken at selected time intervals and transferred from one petri dish to another several times to remove excess water from the hydrogel surface, and then weighed (m_{wet gel}) to determine the swelling of the gels.

Degradation tests: To determine the degradation behavior of the hydrogels, gels were soaked in PBS (pH 7.4) and allowed to sit in an incubator at 37° C. The hydrogels were taken at selected time intervals, lyophilized, and weighed to determine the degradation percentage at different time intervals.

To prepare the copolymer hydrogels, different combinations of two zwitterionic monomers; sulfobetaine methacrylate (SBMA) or carboxybetaine methacrylate (CBMA), and a non-zwitterionic monomer; hydroxyethyl methacrylate (HEMA) were polymerized in the absence of any chemical crosslinker. To initiate polymerization ammonium persulfate (APS) and N,N,N′,N′-Tetramethylethylenediamine (TEMED) couple were used and polymerizations were performed either at room temperature or −20° C. (to prepare cryogels) for 24 h. After polymerization, cryogels were thawed at room temperature.

In initial experiments, the mole ratio of zwitterionic monomer (SBMA or CBMA) to the non-zwitterionic HEMA monomer were kept constant (1:1), but total monomer amounts were varied. All compositions and polymerization conditions are summarized in Table 1.

In general, five different types of materials were obtained after polymerization: a transparent gel, a translucent gel, an opaque gel, a viscous liquid, and a solution (i.e., no gelation).

TABLE 1
Compositions and polymerization conditions of copolymers prepared
1:1 mole ratio of zwitterionic (CBMA or SBMA) and non-zwitterionic
monomer (HEMA). Polymers prepared using pure monomers for control
experiments are also given at the bottom of the table. The materials
were named as follows; the uppercase letters indicate the zwitterionic
monomer (‘S’ or ‘C’ for SBMA or CBMA) and the non-
zwitterionic monomer (‘H’ for HEMA), respectively, that used
in the synthesis. The number indicates the sample number that
prepared using the same monomers and polymerization temperature.
Finally, the lowercase letter(s) indicates the polymerization
temperature (‘rt’ for room temperature and ‘c’ for −20° C.).

| | | | | Tem- | |
|---|---|---|---|---|---|
| | | | | pera- | |
| | SBMA | CBMA | HEMA | ture | |
| Sample | (mg/mL) | (mg/mL) | (mg/mL) | (° C.) | Appearance |
| SH1c | 60 | — | 27 | −20 | Gel (translucent) |
| SH2c | 90 | — | 40.5 | −20 | Gel (translucent) |
| SH3c | 150 | — | 67.5 | −20 | Gel (transparent) |
| SH1rt | 60 | — | 27 | 20 | Solution |
| SH2rt | 90 | — | 40.5 | 20 | Solution |
| SH3rt | 150 | — | 67.5 | 20 | Viscous liquid |
| CH1c | — | 24 | 13.5 | −20 | Gel (translucent) |
| CH2c | — | 48 | 27 | −20 | Gel (translucent) |
| CH3c | — | 72 | 40.5 | −20 | Gel (translucent) |
| CH4c | — | 144 | 81 | −20 | Gel (transparent) |
| CH1rt | — | 24 | 13.5 | 20 | Solution |
| CH2rt | — | 48 | 27 | 20 | Solution |
| CH3rt | — | 72 | 40.5 | 20 | Solution |
| CH4rt | — | 144 | 81 | 20 | Gel (transparent) |
| S1c | — | 150 | — | −20 | Solution |
| C1c | — | 144 | — | −20 | Gel (transparent) |
| H1c | — | — | 81 | −20 | Gel (Opaque) |

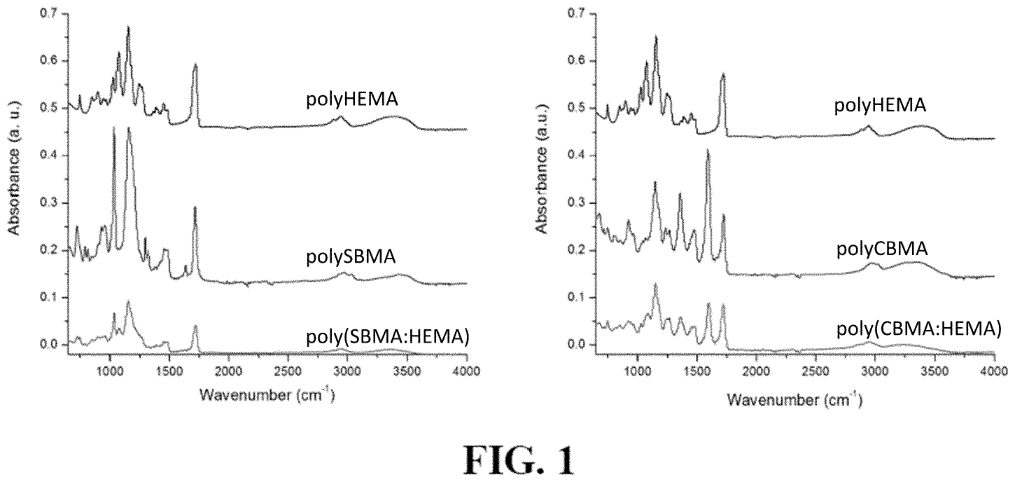

Because no chemical crosslinker was used, the gel formation can be attributed to the strong physical interactions between the zwitterionic side groups of the polymers. In addition, hydroxyl side groups of the polymer can form hydrogen bonds which can further improve the interactions between two polymer chains. Interestingly, all the formulations (Table 1) that were polymerized at cryoconditions (−20° C.) formed gels, but when the same compositions were polymerized at room temperature gel formation was only observed for the CH4rt sample, where high amounts of CBMA (144 mg/mL) were used. For the SBMA monomer, room temperature polymerization did not yield gelation even for the SH3rt sample with a high monomer concentration of 150 mg/mL. For control experiments, we also polymerized solutions that contain only SBMA, CBMA, and HEMA at −20° C. We could not observe gel formation for the SBMA polymerization solutions even at a monomer concentration of 150 mg/mL. Alternatively, CBMA formed gels at a concentration of 144 mg/mL, but the gels were much weaker compared to the gels prepared using both CBMA and HEMA monomers. Polymerization of HEMA solutions (81 mg/mL) also resulted in the formation of an opaque gel. The chemical composition of the polymers and copolymers were characterized using FTIR spectroscopy (FIG. 1). The FTIR adsorption peaks corresponding to both monomers were present in the FTIR spectra demonstrating that both monomers were present in the copolymers.

The gels containing zwitterionic monomers were found to be mechanically stable after stretching or compression. The S3c samples could be stretched to more than 5× their initial size without breaking, and could be highly compressed without any significant deformation.

To determine whether the physical crosslinks of the gels could be reestablished after breakage, we prepared two CH3c or SH3c cryogels, and cut them into two pieces. One of the two gel pieces was colored red using Alizarin Red S dye (25 μg/mL). One colored and one uncolored piece of each gel were then brought together and stretched to determine if they self-healed. We observed almost immediate self-healing for both gels.

Softer cryogels, prepared using lower monomer amounts (for example, the SH1c gel), could be injected from a 21 gauge needle. By comparison, cryogels prepared using only HEMA monomers were highly deformed after compression and could not recover initial shapes after compression. Additionally, self-healing was not observed for the pure HEMA cryogels and they were not injectable. These results suggest that the zwitterionic side groups of the polymer provides good mechanical properties and self-healing abilities to the cryogels.

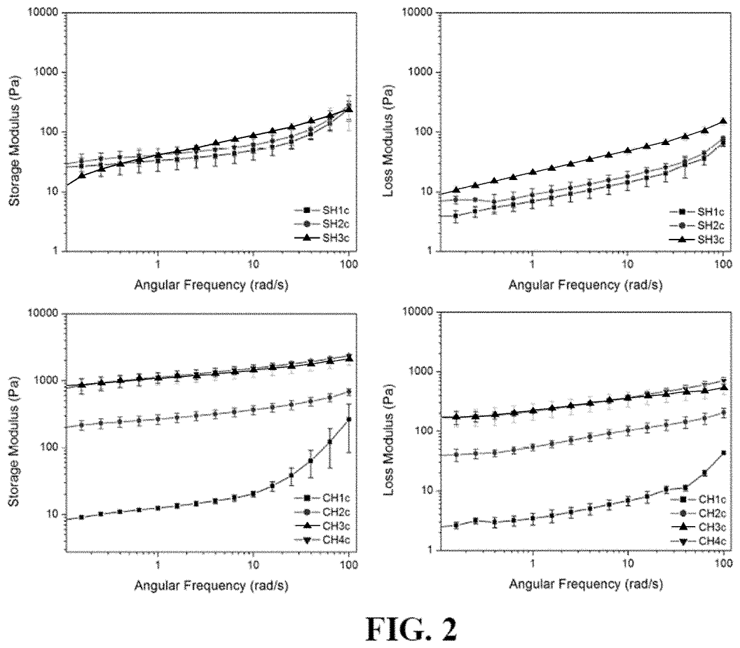

Viscoelastic properties of the cryogels were investigated using rheometry to better evaluate their mechanical properties. FIG. 2 shows the rheology of the cryogels that were prepared using 1:1 mole ratio of SBMA or CBMA, and HEMA monomers. Larger storage modulus (G′) values than loss modulus (G″) values were observed for all of the cryogels indicating their viscoelastic property. In addition, higher G′ and G″ were observed for the CBMA cryogels indicating better mechanical properties of these gels. Stronger gel formation is expected for the gels prepared using the SBMA monomer because it has a greater difference between ionic strengths of the zwitterionic subunits, which should yield stronger ionic interactions between the polymer chains. However, we observed the opposite for the cryogels prepared in this study. It should be noted that other than with increasing ionic strength difference, the physical interactions between polymer chains should become stronger with the increasing average molecular weight of the polymer, which can be the reason for the stronger gel formation with CBMA monomers compared to SBMA monomers.

Higher G′ and G″ values were also observed for the CBMA gels as the total monomer amount was increased up to a CBMA concentration of 72 mg/mL (FIG. 2). Further, increasing the CBMA concentration to 144 mg/mL did not significantly improve the mechanical properties of the cryogels. Alternatively, increasing the SBMA monomer concentration did not significantly affect the mechanical properties of the gels (FIG. 2); there was only a slight improvement in G′ and G″ values even after using a high SBMA concentration of 150 mg/mL.

To understand the effect of monomer mole ratios on the mechanical properties of the hydrogels, the mole ratio of the monomers was varied while keeping the total monomer amount constant. The polymerized compositions were summarized in Table 2.

TABLE 2
Compositions of the copolymers prepared
using different mole ratios of monomers

| | | | | Tem- | |
|---|---|---|---|---|---|
| | | | | pera- | |
| | SBMA | CBMA | HEMA | ture | |
| Sample | (mg/mL) | (mg/mL) | (mg/mL) | (° C.) | Appearance |
| CH5c | — | 108 | 20.3 | −20 | Gel (translucent) |
| (75% | | | | | |
| CBMA) | | | | | |
| CH6c | — | 36 | 60.8 | −20 | Gel (opaque) |
| (25% | | | | | |
| CBMA) | | | | | |
| CH5rt | — | 108 | 20.3 | 20 | Solution |
| (75% | | | | | |
| CBMA) | | | | | |
| CH6rt | — | 36 | 60.8 | 20 | Solution |
| (25% | | | | | |
| CBMA) | | | | | |
| SH4c | 45 | — | 61 | −20 | Gel (opaque) |
| (25% | | | | | |
| SBMA) | | | | | |
| SH5c | 135 | — | 20.5 | −20 | Solution |
| (75% | | | | | |
| SBMA) | | | | | |

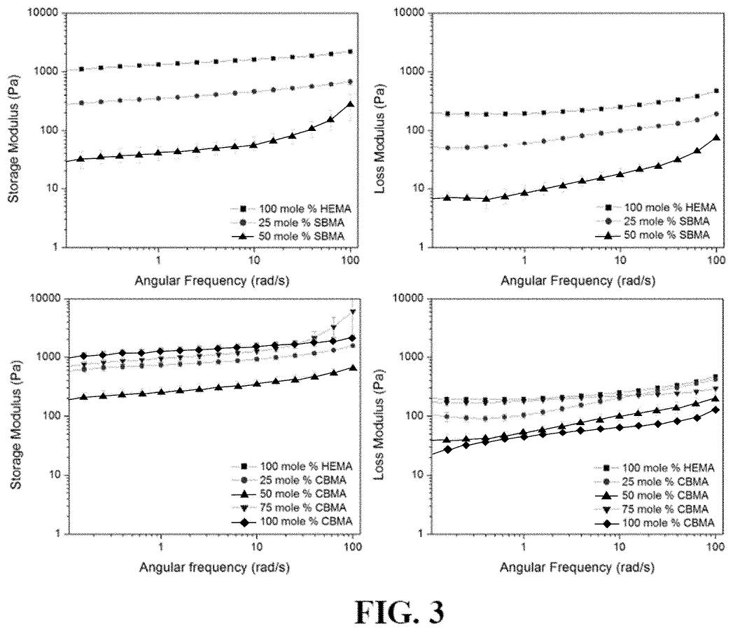

As noted above, the polymerization compositions that contain 100 mole % SBMA monomer did not form gels in any of the tested conditions, but it was possible to prepare hydrogels using pure HEMA and CBMA monomers. Additionally, the solution that contains 75 mole % SBMA did not form a gel, but all other copolymer compositions formed gels when polymerized at cryoconditions. The compositions that yielded hydrogels were then characterized through rheology measurements (FIG. 3). Pure HEMA and CBMA hydrogels showed the highest and lowest G′ and G″, respectively, and copolymer hydrogels demonstrated mechanical properties between those of pure hydrogels. In general, it was observed that increasing the HEMA monomer mole percentage resulted in higher G′ and G″ values. The only exception was the hydrogel prepared using 75 mole % CBMA, which yielded higher G′ and G″ values than 50 mole % CBMA containing hydrogels.

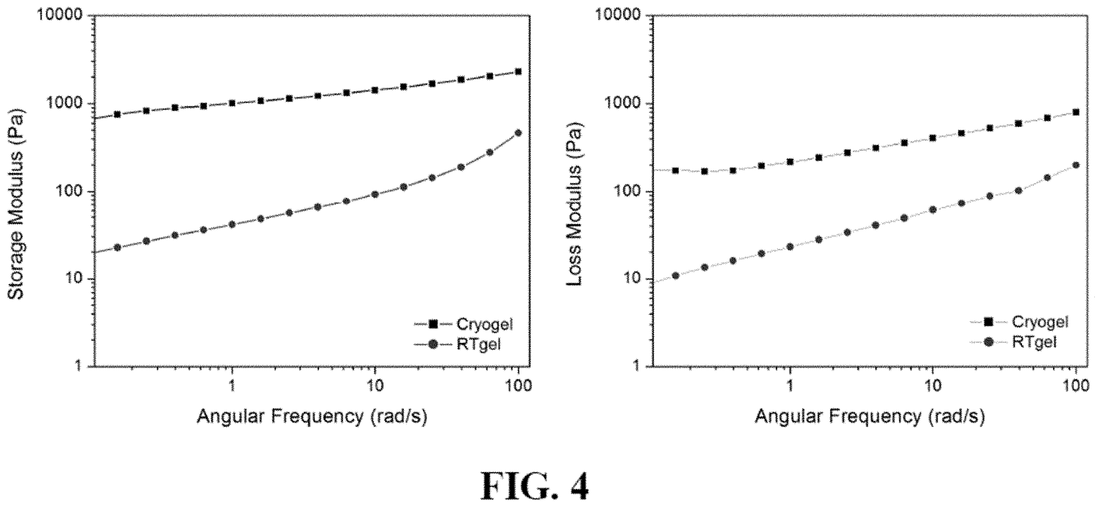

We also performed rheology characterization of the only composition that yielded hydrogel formation using room temperature polymerization, CH4rt, and compared it with its cryogel counterpart (FIG. 4). While the gels formed at room temperature were very weak, around 10-fold higher G′ and G″ values were measured for the same composition polymerized using cryoconditions, indicating that polymerization under cryoconditions improves the mechanical properties of the gels. There are several possible reasons for the improved mechanical properties for the gels polymerized at cryoconditions compared to those prepared at room temperature. First, the cryoconcentration effect, which is locally enhanced monomer concentrations due to the phase separation of the monomer phase and water phase during freezing, may result in the formation of a denser polymer network than their counterparts, which were prepared at room temperature. Second, in addition to a denser network at cryoconditions, longer polymer chains (higher average molecular weight) may be formed due to the reduced polymerization rate, which can improve the interactions between polymer chains and, thus, improve the mechanical properties of the gels. Finally, the freeze-thaw process may improve the mechanical properties of the gels as observed previously for PVA-based physically-crosslinked hydrogels.

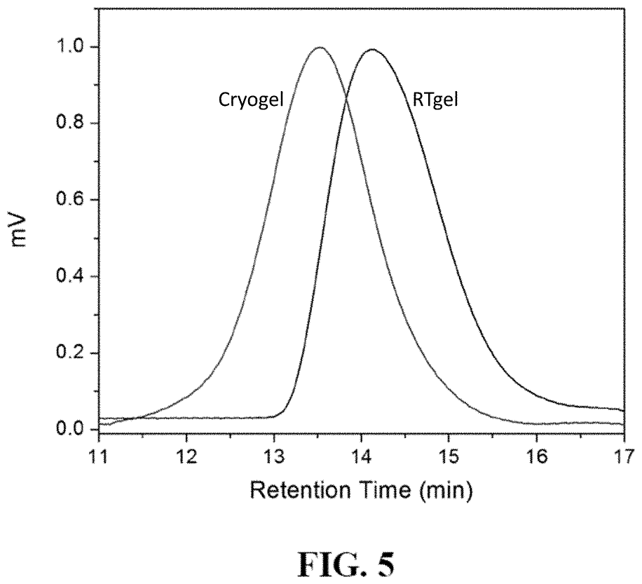

While there is no method to directly observe the cryoconcentration effect, the contribution of the other possible reasons can be tested. To see if the formed chains have higher molecular weights when the same composition was polymerized at lower temperatures, we performed GPC analysis on two poly(SBMA:HEMA) polymers prepared using the same polymerization solution but at different temperatures (room temperature, and −20 C). To prevent gelation, the monomer amounts were kept low (0.04 mg/mL of SBMA and 0.015 mg/mL of HEMA). GPC results (FIG. 5 and Table 3) showed a significantly higher molecular weight for the polymer prepared at cryoconditions.

TABLE 3
GPC results

| | Retention time | Area | Area | | | | |
|---|---|---|---|---|---|---|---|
| | [min] | [mV*sec] | [%] | Mn | Mw | Mz | Mw/Mn |
| CYROGEL | 13.528 | 2927.025 | 100 | 55500 | 100371 | 176746 | 1.808 |
| RT GEL | 14.122 | 2982.973 | 100 | 22110 | 34934 | 48175 | 1.58 |

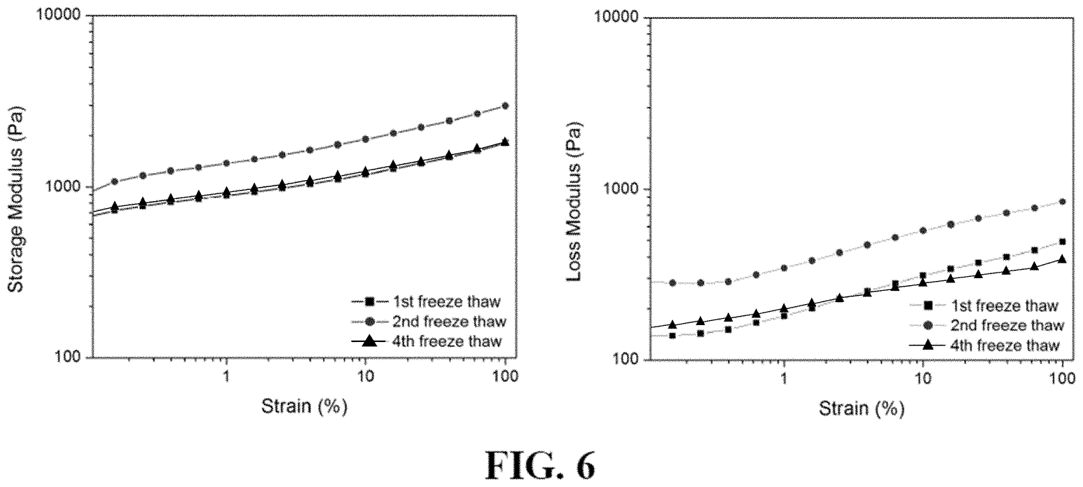

To determine whether the freezing and thawing of the gel contributes to the improved mechanical properties of the cryogels, we performed additional freeze-thaw cycles to CH1c samples and performed rheology measurements afterwards (FIG. 6). After 4 freeze-thaw cycles, there was no significant change in the storage or loss moduli of the gels. These results suggest that the main reason for improved mechanical properties of the cryogels is due to the polymer formation with higher average molecular weight during polymerization, which should yield stronger interactions between the polymer chains. In addition, denser polymer network formation due to the cryoconcentration effect may improve the mechanical properties of the gels.

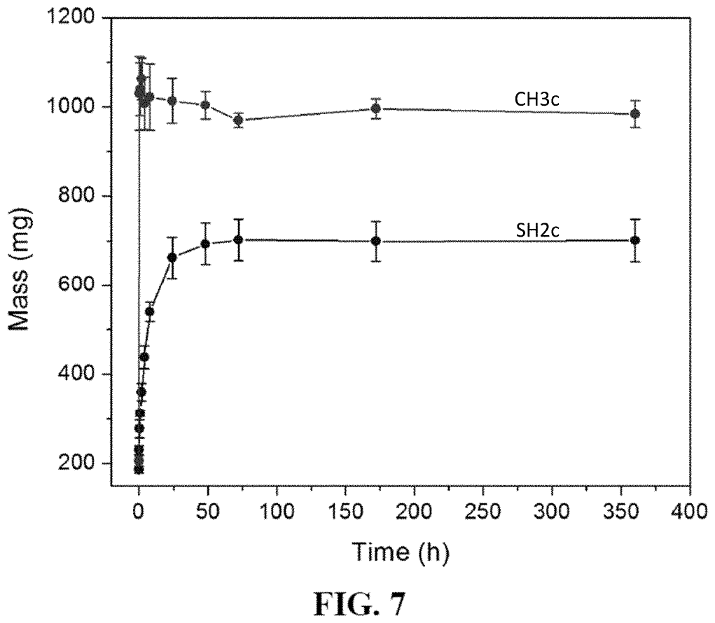

The swelling property of the copolymer cryogels in PBS was also studied (FIG. 7) for SH2c and CH3c samples. The SH2c sample gradually swelled to around 3.5× its initial volume in 2 days and the volume did not change after further incubation in PBS for 13 days. Alternatively, for the CH3c sample, swelling was very rapid. This gel swelled to around 5× of its initial volume in a few minutes and its volume did not change after that for a period of 15 days.

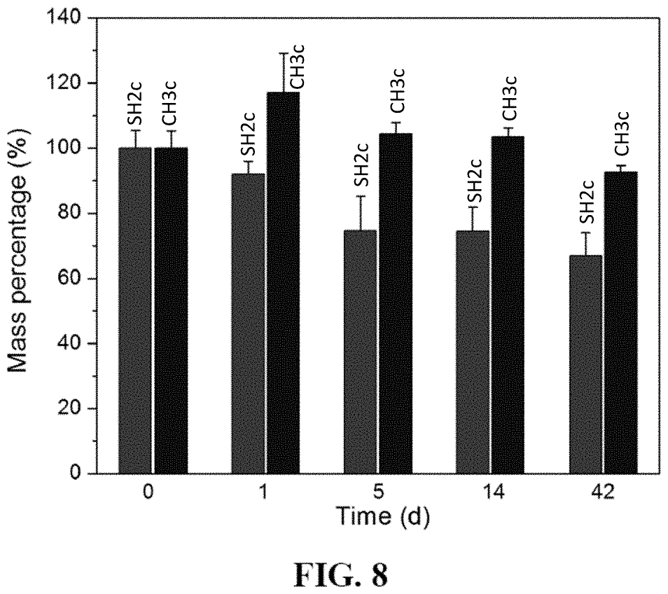

To study the possible degradation of these physically crosslinked hydrogels, the SH2c and CH3c samples were incubated in PBS at 37° C., and at different time intervals, some of the hydrogels were dried and the weight of the dry products was measured. It was observed that after 42 days, the weight loss in the SH2c sample was more than 30%, but only 8% for the CH3c sample (FIG. 8). The lower degradation rate of the CBMA-based hydrogels may be attributed to their stronger interactions between the polymer chains.

### Example 2

Cerium Oxide Nanoparticle with MicroRNA 146a Delivered via Zwitterionic Gel Improves Skin Strength

Impairments in wound healing and wound strength are a significant clinical problem in diabetic wounds. To test whether topical delivery of CNP-miR146a in a zwitterionic gel would be more clinically relevant, resulting in improved healing and improved wound strength, 12-week old female mice breed homozygous diabetic (Db/Db) were given a single, 8mm full thickness punch wound on the dorsal neck skin of each mouse. Zwitterionic gels were prepared by as described above by dissolving SBMA and HEMA in water. CNP-miR146a was added to the gelation solution, and polymerization was initiated using APS and TEMED. The reaction mixtures were poured into a plastic mold with inner diameter of 0.5 cm and polymerized at −20° C. for 24 hr. Cryogels were thawed at room temperature, and unreacted monomers and other unbound ingredients were removed, and the cryogels were washed with PBS several times.

The wounds were treated with one time administration of the hydrogel only (n=5) or the hydrogel impregnated with CNP-miR146a (n=5, about 10 ng). The wounds were photographed over time to closure, and animals were euthanized 4 weeks after wound closure for biomechanical testing. A dumbbell shaped sample was taken from cranial to caudal on each mouse with the healed wound in the center. An Instron 5942 testing unit with Bluehill 3 Software was used for examining maximum load, extension, tensile strain.

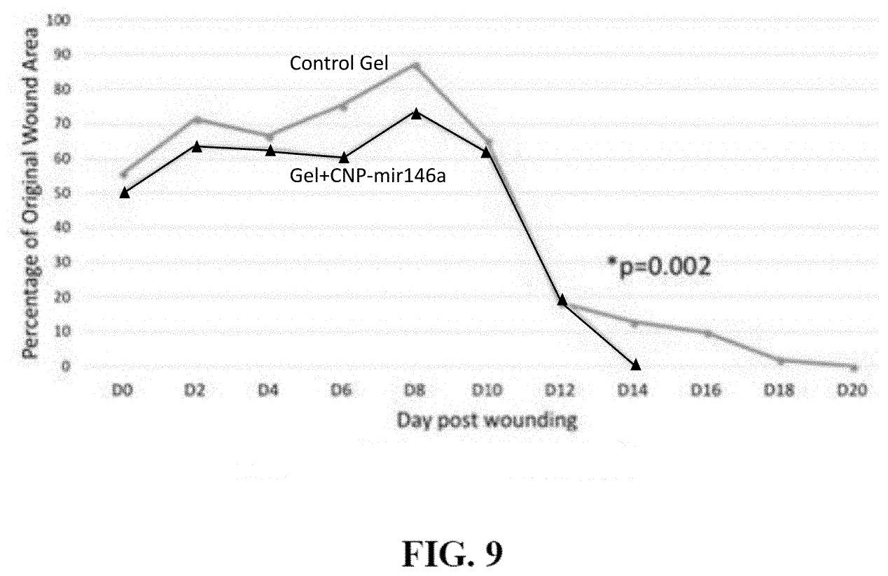

Mice wounds treated with CNP-miR146a hydrogel demonstrated a significant improvement in time to complete wound healing (FIG. 9). Untreated diabetic mouse wounds typically heal at day 22-24 post healing. Wounds treated with the control gel (no CNP-miR146a) healed at day 20 and wounds treated with CNP-miR146a impregnated gel healed at day 14 (P-value=0.002). The wounds also showed improved strength after healing with increased maximum load of 3.24N compared to 2.04N (P-value=0.03).

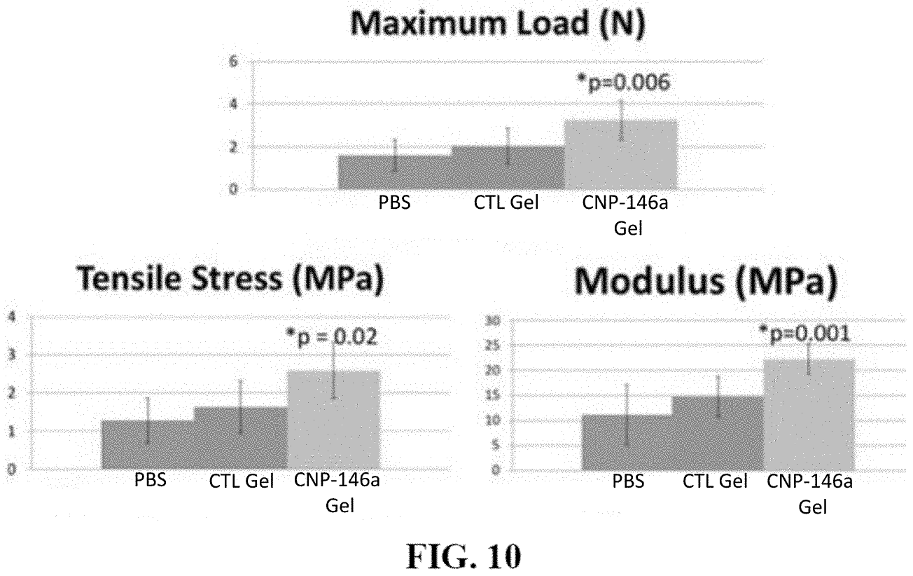

Elastic modulus measures resistance to being deformed when stress is applied. We observed an improved modulus with CNP-146a gel compared to the control gel (22.26 MPa compared to 14.68 MPa; P-value=0.02) (FIG. 10). PBS was also applied as a control. Tensile stress at maximum load is also improved (1.63 MPa in control gel compared to 2.59 in treatment gel; P-value=0.03) (FIG. 10).

These data demonstrate that diabetic mice wounds treated with zwitterionic gel impregnated with CNP-miR146a improved time to complete wound healing, strength, elasticity, and resistance to stress after healing. This study shows the feasibility of topical delivery of a therapeutic for diabetic wound healing via a hydrogel of this disclosure, and no adverse effects on wound strength.

### Example 3

CNP-miR146a Decreases Disease Activity Index in a Murine DSS Colitis Model

Inflammatory bowel disease (IBD), specifically ulcerative colitis, is characterized by intestinal inflammation, oxidative stress, and a disrupted mucosal barrier. To determine whether oxidative stress and inflammation could be targeted using the CNP-146a hydrogels of this disclosure, we first developed a colitis model using 3% DSS to induce colitis in 8-week-old, male C57/BL6 wild type mice for 5 days. We then developed a therapeutic delivery model where mice were treated per rectum with PBS control, 10 ng CNP-miR146a (liquid), lOng CNP-miR146a (hydrogel), 11% viscous silk fibroin (SF) with added lOng CNP-miR146a (SF-viscous), or SF containing CNP-miR146a (SF-liquid).

Once delivery of CNP-miR146a was optimized, mice (n=5) were treated with 20 ng of CNP-miR146a hydrogel for 4 days starting day 5 of DSS administration. They were compared to those treated with a control gel and those receiving no treatment. Disease activity index (DAI) was measured daily.

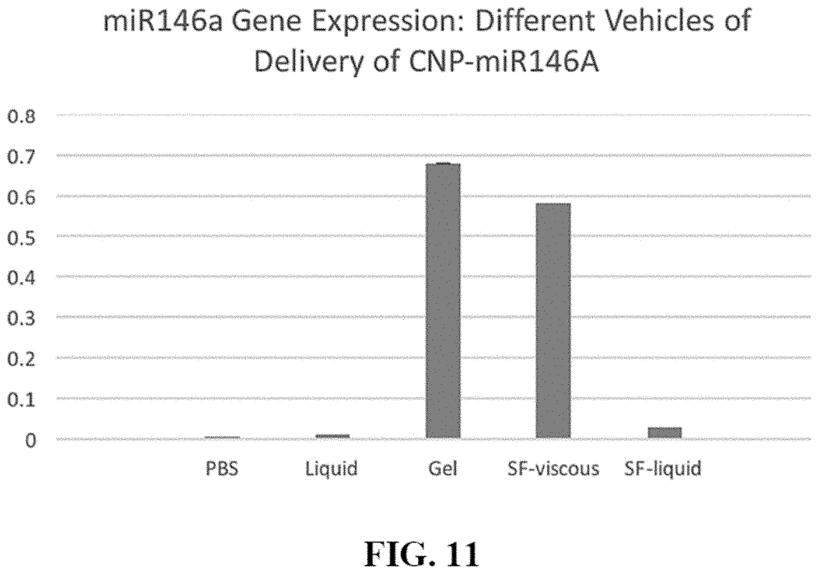

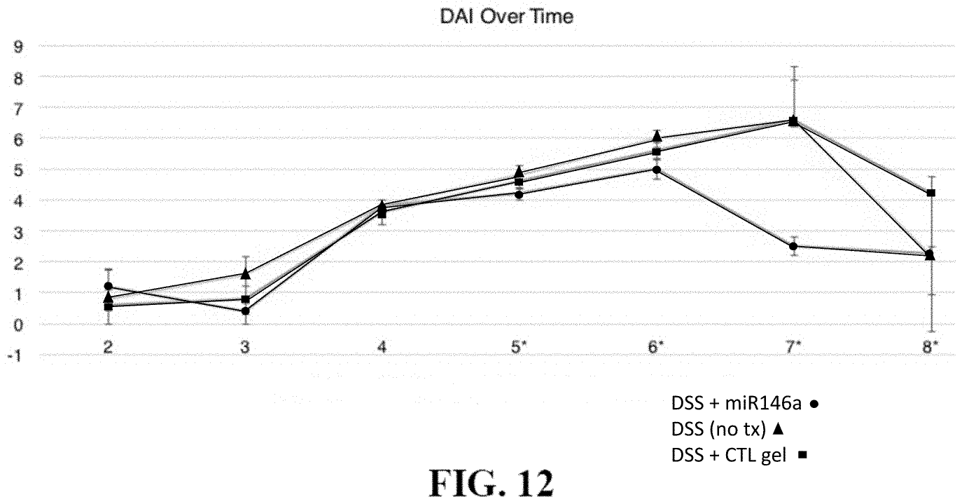

Gene expression demonstrated the highest levels of miR146a using the hydrogel as a delivery vehicle (FIG. 11). Treatment with CNP-miR146a gel resulted in a significantly lower DAI at day 7 (day 3 of treatment, p<0.05) (FIG. 12).

These results demonstrate that in a murine DSS colitis model, the CNP-miR146a hydrogel of this disclosure represents an effective local drug delivery vehicle. Furthermore, the CNP-miR146a gel decreases DAI, demonstrating its potential as a novel therapy for acute colitis.

### Example 4

CM-CNP-miR146a Decreases Weight Loss, Decreases Disease Activity Index, Decreases Colon Length Shortening, and Decreases IL-1b and TNFα Expression in a Murine DSS Colitis Model

To determine whether oxidative stress and inflammation could also be successfully treated using the CNP-146a chitosan microgels of this disclosure, we again utilized a mouse model of colitis. This time, we induced colitis in 14-week-old, male C57/BL6 wild type mice by adding DSS to their drinking water for 6 days. We then developed a novel therapeutic delivery strategy for CNP-miR146a using a chitosan microgel where mice were treated per rectum at day 6 with either PBS (DSS), chitosan microgel alone (DSS+CM), or chitosan microgel containing 500 ng of CNP-miR146a (DSS+CM-CNP-miR146a). The control (CTL) group did not receive DSS or any other treatment. Disease activity index (DAI) was calculated based on percent weight loss, stool consistency, and blood in the stool. The colon was harvested for PCR at day 8 to examine inflammatory gene expression.

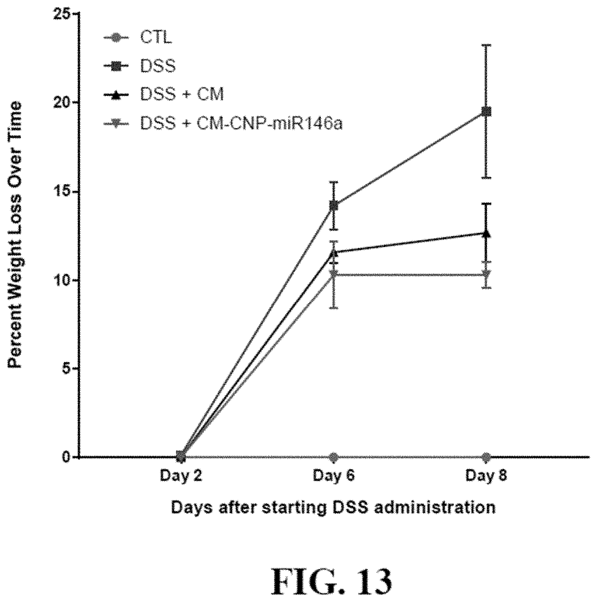

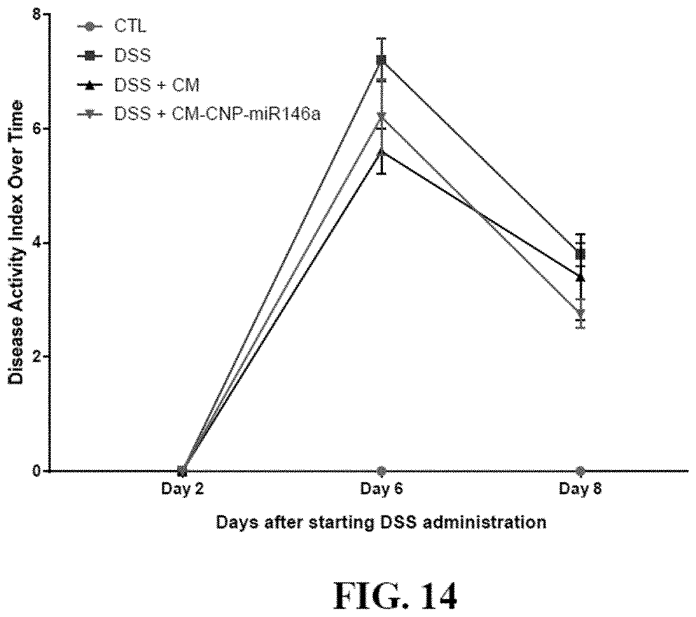

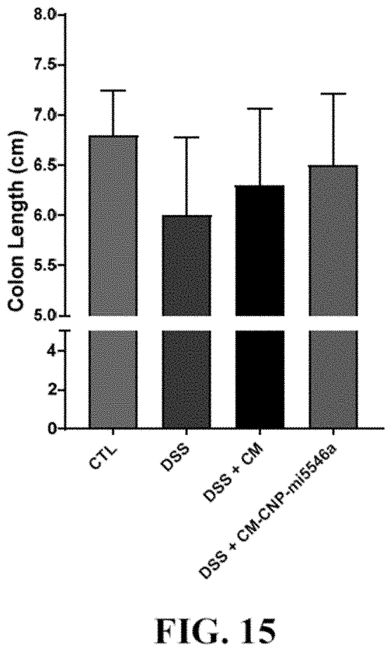

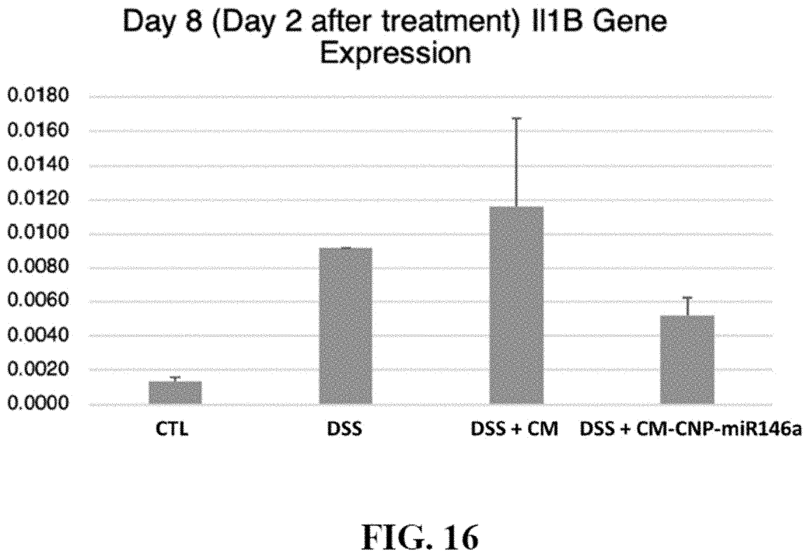

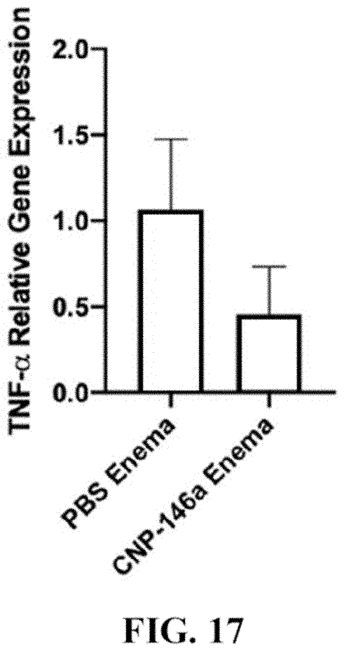

As shown in FIG. 13, treatment with CM-CNP-miR146a resulted in a lower percentage of weight loss over time compared to the mice treated with DSS and DSS+CM. The mice treated with CM-CNP-miR146a also exhibited a significantly lower DAI at day 8 (day 2 after treatment, p<0.05) compared to the PBS treated group (FIG. 14). With treatment of CM-CNP-miR146a, there was also a trend towards a less shortened colon (FIG. 15). The CM-CNP-miR146a-treated mice also demonstrated lower scores related to blood in the stool. Still further, FIG. 16 illustrates that IL-1b gene expression was decreased in the group treated with CM-CNP-miR146a compared to the PBS group (p<0.05). FIG. 17 also shows that TNFα gene expression decreased in DSS mice after treatment with CNP-miR146a chitosan microgel relative to DSS mice treated with chitosan microgel lacking CNP-miR146a.

These results demonstrate that delivery of CNP-miR146a using a chitosan microgel decreases inflammatory gene expression and overall DAI in a murine colitis model. Accordingly, chitosan microgel delivery of CNP-miR146a may provide effective colitis therapy. Furthermore, the CNP-miR146a chitosan microgel decreases DAI, demonstrating its potential as a novel therapy for acute colitis.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, or step is necessary or indispensable. The novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed is:

1. A method for treating inflammation in a subject in need thereof, the method comprising administering to the subject a therapeutically effective amount of a chitosan microgel comprising microRNA-conjugated cerium oxide nanoparticles (CNPs).

2. The method of claim 1, wherein the inflammation is associated with a wound.

3. The method of claim 2, wherein the wound is a diabetic ulcer.

4. The method of claim 2 or 3, wherein the treating results in an increased rate of wound closure in the subject compared to the rate of wound closure in an untreated subject.

5. The method of any one of claims 1-3, wherein the chitosan microgel is administered topically, intradermally, or intramuscularly to the subject.

6. The method of claim 1, wherein the inflammation is associated with ulcerative colitis or Crohn's disease.

7. The method of claim 6, wherein the chitosan microgel is administered orally or rectally to the subject.

8. The method of any one of claims 1-7, wherein the chitosan microgel is administered to the subject a plurality of times.

9. The method of any one of claims 1-8, wherein the chitosan microgel is administered daily to the subject.

10. The method of any of claims 1 to 9, wherein administration of the chitosan microgel treats or prevents oxidative stress in the subject.

11. The method of any of claims 1 to 10, wherein the microRNA comprises miRNA-146a.

12. The method of any of claims 1 to 11, wherein the surface of the CNPs is coated with one or more biocompatible molecules selected from hyaluronic acid, collagen, and fibrinogen.

13. The method of any of claims 1 to 12, wherein the CNPs have a size range of about 3-5 nm.

14. The method of any of claims 1 to 13, wherein the CNPs are doped with a lanthanide selected from one or more of Europium (Eu), Lanthanum (La), Praseodymium (Pr), Neodymium (Nd), Promethium (Pm), Samarium (Sm), Gadolinium (Gd), Terbium (Tb), Dysprosium (Dy), Homium (Ho), Erbium (Er), Thulium (Tm), Ytterbium (Yb), and Lutetium (Lu).

15. A pharmaceutical composition comprising miRNA146a-conjugated cerium oxide nanoparticles (CNPs) embedded within a chitosan microgel.

16. The pharmaceutical composition of claim 15, wherein the surface of the CNPs is coated with one or more biocompatible molecules selected from hyaluronic acid, collagen, and fibrinogen.

17. The pharmaceutical composition of claim 15 or 16, wherein the CNPs have a size range of about 3-5 nm.

18. The pharmaceutical composition of any one of claims 15-17, wherein the CNPs are doped with a lanthanide selected from one or more of Europium (Eu), Lanthanum (La), Praseodymium (Pr), Neodymium (Nd), Promethium (Pm), Samarium (Sm), Gadolinium (Gd), Terbium (Tb), Dysprosium (Dy), Homium (Ho), Erbium (Er), Thulium (Tm), Ytterbium (Yb), and Lutetium (Lu).

19. A microgel comprising:
chitosan polymers; and
a cerium oxide nanoparticle (CNP).

20. The microgel of claim 19, wherein the CNP comprises a therapeutic agent.

21. The microgel of claim 20, wherein the therapeutic agent is an anti-inflammatory agent.

22. The microgel of claim 20 or 21, wherein the therapeutic agent is a micro RNA (miRNA).

23. The microgel of claim 22, wherein the miRNA is miRNA 146a.

24. A method of making an anti-inflammatory chitosan microgel composition, the method comprising:
a) forming a composition comprising a plurality of chitosan polymers and a cerium oxide nanoparticle (CNP); and
b) crosslinking the chitosan polymers with genipin to form a chitosan microgel comprising anti-inflammatory CNP.

25. The method of claim 24, wherein the CNP comprises a therapeutic agent.

26. The method of claim 25, wherein the therapeutic agent is an anti-inflammatory agent.

27. The method of claim 25 or 26, wherein the therapeutic agent is a micro RNA (miRNA).

28. The method of claim 27, wherein the miRNA is miRNA 146a.

29. A method for treating inflammation in a subject in need thereof, the method comprising administering to the subject a therapeutically effective amount of a zwitterionic gel comprising microRNA-conjugated cerium oxide nanoparticles (CNPs).

30. The method of claim 29, wherein the inflammation is associated with a wound.

31. The method of claim 30, wherein the wound is a diabetic ulcer.

32. The method of claim 30 or claim 31, wherein the treating results in an increased rate of wound closure in the subject compared to the rate of wound closure in an untreated subject.

33. The method of any one of claims 29 to 31, wherein the zwitterionic gel is administered topically, intradermally, or intramuscularly to the subject.

34. The method of claim 29, wherein the inflammation is associated with ulcerative colitis or Crohn's disease.

35. The method of claim 34, wherein the zwitterionic gel is administered orally or rectally to the subject.

36. The method of any one of claims 29 to 35, wherein the zwitterionic gel is administered to the subject a plurality of times.

37. The method of any one of claims 29 to 36, wherein the zwitterionic gel is administered daily to the subject.

38. The method of any of claims 29 to 37, wherein administration of the zwitterionic gel treats or prevents oxidative stress in the subject.

39. The method of any of claims 29 to 38, wherein the zwitterionic gel is a zwitterionic cryogel.

40. The method of any of claims 29 to 39, wherein the zwitterionic gel comprises [2-(methacryloyloxy)ethyl]dimethyl-(3-sulfopropyl)ammonium hydroxide (SBMA) and/or 2-hydroxyethyl methacrylate (HEMA) monomers.

41. The method of any of claims 29 to 40, wherein the microRNA comprises miRNA-146a.

42. The method of any of claims 29 to 41, wherein the surface of the CNPs is coated with one or more biocompatible molecules selected from hyaluronic acid, collagen, and fibrinogen.

43. The method of any of claims 29 to 42, wherein the CNPs have a size range of about 3-5 nm.

44. The method of any of claims 29 to 43, wherein the CNPs are doped with a lanthanide selected from one or more of Europium (Eu), Lanthanum (La), Praseodymium (Pr), Neodymium (Nd), Promethium (Pm), Samarium (Sm), Gadolinium (Gd), Terbium (Tb), Dysprosium (Dy), Homium (Ho), Erbium (Er), Thulium (Tm), Ytterbium (Yb), and Lutetium (Lu).

45. A pharmaceutical composition comprising miRNA146a-conjugated cerium oxide nanoparticles (CNPs) embedded within a zwitterionic hydrogel.

46. The pharmaceutical composition of claim 45, wherein the surface of the CNPs is coated with one or more biocompatible molecules selected from hyaluronic acid, collagen, and fibrinogen.

47. The pharmaceutical composition of claim 45 or 46, wherein the CNPs have a size range of about 3-5 nm.

48. The pharmaceutical composition of any one of claims 45 to 47, wherein the CNPs are doped with a lanthanide selected from one or more of Europium (Eu), Lanthanum (La), Praseodymium (Pr), Neodymium (Nd), Promethium (Pm), Samarium (Sm), Gadolinium (Gd), Terbium (Tb), Dysprosium (Dy), Homium (Ho), Erbium (Er), Thulium (Tm), Ytterbium (Yb), and Lutetium (Lu).

49. A hydrogel comprising:
a) polymerized zwitterionic monomers selected from the group consisting of sulfobetaine methacrylate (SBMA), carboxybetaine methacrylate (CBMA), and combinations thereof;
b) polymerized hydroxyethyl methacrylate (HEMA) monomers; and
c) a cerium oxide nanoparticle (CNP).

50. The hydrogel of claim 49, wherein the CNP comprises a therapeutic agent.

51. The hydrogel of claim 50, wherein the therapeutic agent is an anti-inflammatory agent.

52. The hydrogel of claim 50 or 51, wherein the therapeutic agent is a micro RNA (miRNA).

53. The hydrogel of claim 52, wherein the miRNA is miRNA-146a.

54. A method of making an anti-inflammatory hydrogel composition, comprising:
a) forming a composition comprising at least one zwitterionic monomer selected from the group consisting of sulfobetaine methacrylate (SBMA) and carboxybetaine methacrylate (CBMA); hydroxyethyl methacrylate (HEMA) monomers; and a cerium oxide nanoparticle (CNP);
b) initiating the polymerization of the monomers in the composition by the addition of a chemical polymerizing agent to the composition; and
c) polymerizing the monomers in the composition at a temperature below 0° C. to form a hydrogel comprising anti-inflammatory CNP.

55. The method of claim 54, wherein the CNP comprises a therapeutic agent.

56. The method of claim 55, wherein the therapeutic agent is an anti-inflammatory agent.

57. The method of claim 55 or 56, wherein the therapeutic agent is a micro RNA (miRNA).

58. The method of claim 57, wherein the miRNA is miRNA-146a.

59. The method of claim 54, wherein the polymerizing agent comprises ammonium persulfate (APS) and N,N,N′,N′-Tetramethylethylenediamine (TEMED).

60. The method of claim 54, wherein the step of polymerizing the monomers in the composition is conducted at a temperature of about −20° C.


SEQUENCE LISTING (1 sequence)

SEQ ID NO: 1 - RNA, 22 nt, Homo sapiens
ugagaacugaauuccauggguu